(19) United States
(12) Patent Application Publication
(10) Pub. No.: US 2024/0026429 A1
(43) Pub. Date: Jan. 25, 2024

(54) BUFFER COMPOSITIONS FOR PROCESSING OF BIOLOGICAL SAMPLES

(71) Applicant: Quidel Corporation, San Diego, CA (US)

(72) Inventor: Julius Maru de Leon, San Diego, CA (US)

(21) Appl. No.: 18/224,535

(22) Filed: Jul. 20, 2023

Related U.S. Application Data
(60) Provisional application No. 63/368,959, filed on Jul. 20, 2022.

(51) Int. Cl. C12Q 1/6844; C12N 15/10

(52) U.S. Cl. CPC C12Q 1/6844; C12N 15/1013; C12Q 1/689

(57) ABSTRACT

Buffer compositions, such as two-part buffer compositions and buffer systems, methods, assays, kits and kits of parts comprising buffers, for rapid, non-hazardous and fully automated extraction of nucleic acids from various samples. The described buffers do not contain hazardous materials, yet unexpectedly provide more efficient nucleic acid extraction when compared to standard buffers containing harmful ingredients. The present technology provides superior nucleic acid extraction efficiency without the drawbacks associated with standard, hazardous extraction buffers.

### CROSS REFERENCE TO RELATED APPLICATIONS

This application claims the benefit of U.S. Provisional Patent Application No. 63/368,959, filed on Jul. 20, 2022, which is incorporated herein by reference in its entirety.

### TECHNICAL FIELD

The subject matter described herein relates to buffer compositions, such as two-part buffer compositions and buffer systems, methods, assays, kits and kits of parts comprising buffers, for rapid, non-hazardous and fully automated extraction of nucleic acids from various samples, such as samples suspected of containing pathogenic viruses, bacterium and the like. The buffers, methods and kits provided herein, for example, do not contain hazardous materials, and provide more efficient nucleic acid extraction when compared to standard buffers containing harmful ingredients. The buffers, methods and kits described herein, for example, provide less complicated and more rapid nucleic acid extraction protocols when compared to the extended, complex and involved methodologies associated with using standard extraction buffers. Accordingly, the technology described herein provides superior nucleic acid extraction efficiency without the drawbacks associated with standard, hazardous extraction buffers.

### BACKGROUND

In the analysis of a sample, determining presence of certain nucleic acids, such as nucleic acids specific for certain microbes, is desirable for a high level of accuracy and sensitivity. The availability of amplification techniques, such as polymerase chain reaction (PCR) and other nucleic acid amplification technologies, make nucleic acid detection and differentiation a sensitive technique for analysis of a pathogen or other agent in a sample of biological origin. However, extraction of nucleic acids from various samples can be difficult and problematic, requiring the use of toxic buffers containing hazardous materials in complicated, time-consuming extraction methods. There remains a need for non-hazardous buffer compositions for safe and rapid extraction of nucleic acids. The buffers described herein unexpectedly provide superior extraction efficiency without hazardous ingredients and without requiring complicated, time-consuming protocols.

### BRIEF SUMMARY

The following aspects and embodiments thereof, described and illustrated below, are meant to be exemplary and illustrative, not limiting in scope.

In a first aspect, the present technology is related to a two-part buffer composition, comprising (i) a first container comprising a lysis buffer comprising a lysis reagent, an antioxidant, and a detergent, the lysis buffer having a pH of about 1.8 to about 3.0; and (ii) a second container comprising a wash buffer comprising a wash reagent and a detergent, the wash buffer having a pH of about 6.8 to about 7.6; wherein the two part buffer composition comprises a nucleic acid extraction buffer system.

In another aspect, the present technology is related to a method for extracting nucleic acid from a sample, comprising providing a sample suspected of comprising nucleic acid; contacting the sample with a lysis buffer having a pH of about 1.8 to about 3.0, the lysis buffer comprising a lysis reagent, an antioxidant, and a lysis detergent, to produce a lysed sample comprising extracted nucleic acid, if present; contacting the lysed sample with a plurality of magnetic particles to produce magnetic particles complexed with the extracted nucleic acid, if present; exposing the magnetic particles complexed with the extracted nucleic acid, if present, with a magnetic field and removing the lysis buffer; contacting the magnetic particles complexed with the extracted nucleic acid, if present, with a wash buffer having a pH of about 6.8 to about 7.6, the wash buffer comprising a wash reagent and a wash detergent, to produce a washed sample; exposing the washed sample comprising magnetic particles complexed with the nucleic acid, if present, with a magnetic field and removing the wash buffer to produce magnetic particles complexed with washed nucleic acid, if present; contacting the magnetic particles complexed with washed nucleic acid, if present, with an elution buffer to produce an elution sample comprising washed, extracted nucleic acid, if present; exposing the elution sample comprising washed, extracted nucleic acid, if present, to a magnetic field to remove the magnetic particles; contacting the eluted, washed, extracted nucleic acid, if present, with reagents for amplification of the nucleic acid; and amplifying the nucleic acid, if present, and detecting its amplification products.

In another aspect, the present technology is related to a method to isolate a nucleic acid from a sample, comprising providing an assay comprised of a cartridge with a port for receiving a biological sample; providing or instructing to obtain a lysis buffer having a pH of about 1.8 to about 3.0, the lysis buffer comprising or consisting essentially of about 47.5 wt % n-acetyl cysteine (NAC), about 0.1 wt % polyoxyethylene acyl ether (Brij-58), about 1.5 wt % sodium citrate pH 2.5, and about 0.5 wt % aluminum potassium sulfate; and providing or instructing to obtain a wash buffer having a pH of about 6.8 to about 7.6, the wash buffer comprising or consisting essentially of 0.5 wt % potassium citrate and 0.01 wt % a polysorbate-type nonionic surfactant (Tween-20).

In another aspect, the present technology is related to a method to isolate a nucleic acid from a sample, comprising providing an assay comprised of a cartridge with a port for receiving a biological sample; providing or instructing to obtain a lysis buffer having a pH of about 1.8 to about 3.0, the lysis buffer comprising or consisting essentially of about 47.5 wt % NAC, about 0.1 wt % polyoxyethylene acyl ether (Brij-58), about 1.5 wt % sodium acetate pH 3.5, and about 0.5 wt % aluminum potassium sulfate; and providing or instructing to obtain a wash buffer having a pH of about 6.8 to about 7.6, the wash buffer comprising or consisting essentially of 0.5 wt % potassium citrate and 0.01 wt % a polysorbate-type nonionic surfactant (Tween-20).

In another aspect, the present technology is related to a method to isolate a nucleic acid from a sample, comprising: providing an assay comprised of a cartridge with a port for receiving a biological sample; providing or instructing to obtain a lysis buffer having a pH of about 1.8 to about 3.0, the lysis buffer comprising or consisting essentially of about 47.5 wt % NAC, about 0.1% polyoxyethylene acyl ether (Brij-58), about 1.5 wt % glycine pH 3.2, and about 0.5 wt % aluminum potassium sulfate; and providing or instructing to obtain a wash buffer having a pH of about 6.8 to about 7.6, the wash buffer comprising or consisting essentially of 0.5 wt % potassium citrate and 0.01 wt % a polysorbate-type nonionic surfactant (Tween-20).

In another aspect, the present technology is related to a method to isolate a nucleic acid from a sample, comprising: providing an assay comprised of a cartridge with a port for receiving a biological sample; providing or instructing to obtain a lysis buffer having a pH of about 1.8 to about 3.0, the lysis buffer comprising or consisting essentially of about 47.5 wt % NAC, about 0.1 wt % polyoxyethylene acyl ether (Brij-58), about 1.5 wt % PBS plus 0.5M NaCl pH 2.6, and about 0.5 wt % aluminum potassium sulfate; and providing or instructing to obtain a wash buffer having a pH of about 6.8 to about 7.6, the wash buffer comprising or consisting essentially of 0.5 wt % potassium citrate and 0.01 wt % a polysorbate-type nonionic surfactant (Tween-20).

In another aspect, the present technology is related to an assay for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % sodium citrate pH 2.5, and about 0.5 wt % aluminum potassium sulfate; wherein the wash buffer comprises or consists essentially of 0.5 wt % potassium citrate and 0.01 wt % a polysorbate-type nonionic surfactant (Tween-20); and wherein the assay platform comprises a port for receiving a biological sample and a chamber that receives or contains the lysis buffer.

In another aspect, the present technology is related to an assay for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % sodium acetate pH 3.5, and about 0.5 wt % aluminum potassium sulfate; wherein the wash buffer comprises or consists essentially of about 0.5 wt % potassium citrate and 0.01 wt % a polysorbate-type nonionic surfactant (Tween-20); and wherein the assay platform comprises a port for receiving a biological sample and a chamber that receives or contains the lysis buffer.

In another aspect, the present technology is related to an assay for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % glycine pH 3.2, and about 0.5 wt % aluminum potassium sulfate; wherein the wash buffer comprises or consists essentially of about 0.5 wt % potassium citrate and 0.01 wt % a polysorbate-type nonionic surfactant (Tween-20); and wherein the assay platform comprises a port for receiving a biological sample and a chamber that receives or contains the lysis buffer.

In another aspect, the present technology is related to an assay for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % PBS plus 0.5M NaCl pH 2.6, and about 0.5 wt % aluminum potassium sulfate; wherein the wash buffer comprises or consists essentially of about 0.5 wt % potassium citrate and 0.01 wt % a polysorbate-type nonionic surfactant (Tween-20); and wherein the assay platform comprises a port for receiving a biological sample and a chamber that receives or contains the lysis buffer.

In another aspect, the present technology is related to a kit comprising a two-part buffer composition as described herein and user instructions for extracting, washing and eluting nucleic acid from a sample with said two-part buffer composition. In some embodiments, the kits described herein include a container and a pipette.

In another aspect, the present technology is related to a kit of parts for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % sodium citrate pH 2.5, and about 0.5 wt % aluminum potassium sulfate; and wherein the wash buffer comprises or consists essentially of about 0.5 wt % potassium citrate and 0.01 wt % a polysorbate-type nonionic surfactant (Tween-20).

In another aspect, the present technology is related to a kit of parts for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % sodium acetate pH 3.5, and about 0.5 wt % aluminum potassium sulfate; and wherein the wash buffer comprises or consists essentially of about 0.5 wt % potassium citrate and 0.01 wt % a polysorbate-type nonionic surfactant (Tween-20).

In another aspect, the present technology is related to a kit of parts for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % glycine pH 3.2, and about 0.5 wt % aluminum potassium sulfate; and wherein the wash buffer comprises or consists essentially of about 0.5 wt % potassium citrate and 0.01 wt % a polysorbate-type nonionic surfactant (Tween-20).

In another aspect, the present technology is related to a kit of parts for isolation and/or detection of a nucleic acid, comprising: an assay platform, a lysis buffer having a pH of about 1.8 to about 3.0, and a wash buffer having a pH of about 6.8 to about 7.6; wherein the lysis buffer comprises or consists essentially of about 47.5 wt % NAC, about 0.1 wt % polyoxyethylene acyl ether (Brij-58), about 1.5 wt % PBS plus 0.5M NaCl pH 2.6, and about 0.5 wt % aluminum potassium sulfate; and wherein the wash buffer comprises or consists essentially of about 0.5 wt % potassium citrate and 0.01 wt % a polysorbate-type nonionic surfactant (Tween-20).

In embodiments, the two-part buffer compositions, methods, assays, kits and kits of parts described herein include a lysis reagent comprising a citrate compound. In embodiments, the lysis reagent comprises sodium citrate. In embodiments, the lysis reagent comprises sodium citrate present in the lysis buffer in a range of between about 0.5-2.5 wt % or between 1.0-2.0 wt %. In embodiments, the lysis reagent comprises an acetate compound. In embodiments, the lysis reagent comprises sodium acetate. In embodiments, the lysis reagent comprises sodium acetate present in the lysis buffer in a range of between about 0.5-2.5 wt % or between 1.0-2.0 wt %. In embodiments, the lysis reagent comprises a glycine compound. In embodiments, the lysis reagent comprises glycine, optionally present in the lysis buffer in a range of between about 1-2 wt %. In embodiments, the lysis reagent comprises phosphate buffered saline (PBS) and sodium chloride (NaCl). In embodiments, the lysis reagent comprises PBS and 0.5M NaCl at a pH of between about 2.4-2.8 or optionally at pH 2.6. In embodiments, the lysis reagent comprises about 1.5 wt % PBS and 0.5M NaCl at pH 2.6.

In embodiments, the two-part buffer compositions, methods, assays, kits and kits of parts described herein comprise an antioxidant comprising n-acetyl cysteine (NAC). In embodiments, the antioxidant comprises NAC present in the lysis buffer in a range of between about 40-55 wt % or between about 45-50 wt %.

In embodiments, the two-part buffer compositions, methods, assays, kits and kits of parts described herein comprise a detergent in the lysis buffer is a non-ionic surfactant. In embodiments, the detergent in the lysis buffer comprises a non-ionic polyoxyethylene surfactant, optionally present in the lysis buffer in a range of between about 0.025-0.2 wt %.

In embodiments, the two-part buffer compositions, methods, assays, kits and kits of parts described herein comprise a lysis buffer comprising aluminum. In embodiments, the lysis buffer comprises aluminum potassium sulfate. In embodiments, the lysis buffer comprises about 0.5 wt % aluminum potassium sulfate.

In embodiments, the two-part buffer compositions, methods, assays, kits and kits of parts described herein comprise a wash reagent comprising a citrate compound. In embodiments, the wash reagent comprises potassium citrate. In embodiments, the wash reagent comprises potassium citrate present in the wash buffer in a range of between about 0.1-1.5 wt % or between about 0.25-1.0 wt %. In embodiments, the wash detergent comprises a polysorbate surfactant (e.g., a Tween®). In embodiments, the wash detergent comprises Tween®-20. In embodiments, the wash detergent comprises a polysorbate-type nonionic surfactant (Tween-20) present in the wash buffer in a range of between about 0.001-0.1 wt % or between about 0.005-0.05 wt %.

In embodiments, the two-part buffer compositions, methods, assays, kits and kits of parts described herein comprise a lysis reagent comprising sodium citrate, sodium acetate, glycine, or PBS plus NaCl; an antioxidant comprising NAC; a lysis detergent comprising Brij®-58 and aluminum potassium sulfate; and a wash reagent comprising a citrate reagent and a polysorbate-type nonionic surfactant (e.g., a Tween®).

In embodiments, the two-part buffer compositions, methods, assays, kits and kits of parts described herein comprise a lysis reagent comprising about 1.5 wt % sodium citrate, about 1.5 wt % sodium acetate, about 1.5 wt % glycine pH 3.2, or about 1.5 wt % PBS plus NaCl pH 2.6; an antioxidant comprising about 47.5 wt % NAC; a lysis detergent comprising about 0.1 wt % polyoxyethylene acyl ether (Brij-58) and about 0.5 wt % aluminum potassium sulfate; and a wash reagent comprising about 0.5 wt % potassium citrate and about 0.01 wt % a polysorbate-type nonionic surfactant (Tween-20).

In embodiments, the two-part buffer compositions, methods, assays, kits and kits of parts described herein the nucleic acid extraction buffer system comprises magnetic particles.

In embodiments, the two-part buffer compositions, methods, assays, kits and kits of parts described herein comprise a cartridge and an instrument for automated extraction, washing, elution and amplification of nucleic acid, if present.

In addition to the exemplary aspects and embodiments described above, further aspects and embodiments will become apparent by reference to the drawings and by study of the following descriptions.

Additional embodiments of the present compositions, methods, kits and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE FIGURES

FIG. 1 illustrates SARS-CoV-2 nucleotide extraction efficiency of citrate containing buffer compositions of the present technology compared to standard extraction buffers, assessed by PCR sensitivity and detection. Nucleotide extraction with citrate buffers of the present technology provides better PCR sensitivity and detection of SARS-CoV-2 than standard buffers.

FIG. 2 illustrates Flu A nucleotide extraction efficiency of citrate containing buffer compositions of the present technology compared to standard extraction buffers, assessed by PCR sensitivity and detection. Nucleotide extraction with citrate buffers of the present technology provides comparable PCR sensitivity and detection of FluA than standard buffers.

FIG. 3 illustrates Flu B nucleotide extraction efficiency of citrate containing buffer compositions of the present technology compared to standard extraction buffers, assessed by PCR sensitivity and detection. Nucleotide extraction with citrate buffers of the present technology provides better PCR sensitivity and detection of FluB than standard buffers.

FIG. 4 illustrates RSV-A nucleotide extraction efficiency of citrate containing buffer compositions of the present technology compared to standard extraction buffers, assessed by PCR sensitivity and detection. Nucleotide extraction with citrate buffers of the present technology provides better PCR sensitivity and detection of RSV-A than standard buffers.

FIG. 5 illustrates SARS-CoV-2 nucleotide extraction efficiency of citrate containing buffer compositions of the present technology compared to standard extraction buffers, assessed by PCR sensitivity and detection on a real-time PCR platform (Savanna®, Quidel Corporation). Nucleotide extraction with citrate buffers of the present technology provides better PCR sensitivity and detection of SARS-CoV-2 than standard buffers.

FIG. 6 illustrates SARS-CoV-2 nucleotide extraction efficiency of citrate, sodium acetate, glycine, and PBS+NaCl containing buffer compositions of the present technology, assessed by PCR sensitivity and detection on a real-time PCR platform (Savanna®, Quidel Corporation). Nucleotide extraction with sodium acetate, glycine, and PBS+NaCl containing buffer compositions of the present technology provides comparable PCR sensitivity and detection of SARS-CoV-2 as seen with citrate buffers of the present technology.

FIG. 7 illustrates Flu A nucleotide extraction efficiency of citrate, sodium acetate, glycine, and PBS+NaCl containing buffer compositions of the present technology, assessed by PCR sensitivity and detection on a real-time PCR platform (Savanna®, Quidel Corporation). Nucleotide extraction with sodium acetate, glycine, and PBS+NaCl containing buffer compositions of the present technology provides comparable PCR sensitivity and detection of FluA as seen with citrate buffers of the present technology.

FIG. 8 illustrates Enterovirus nucleotide extraction efficiency of citrate, sodium acetate, glycine, and PBS+NaCl containing buffer compositions of the present technology, assessed by PCR sensitivity and detection on real-time PCR platform (Savanna®, Quidel Corporation). Nucleotide extraction with sodium acetate, glycine, and PBS+NaCl containing buffer compositions of the present technology provides comparable PCR sensitivity and detection of Enterovirus as seen with citrate buffers of the present technology.

FIG. 9 illustrates Adenovirus nucleotide extraction efficiency of citrate, sodium acetate, glycine, and PBS+NaCl containing buffer compositions of the present technology, assessed by PCR sensitivity and detection on real-time PCR platform (Savanna®, Quidel Corporation). Nucleotide extraction with citrate buffers of the present technology provides better PCR sensitivity and detection of Adenovirus than is seen with sodium acetate, glycine, and PBS+NaCl containing buffer compositions of the present technology.

FIG. 10 illustrates SARS-CoV-2 nucleotide extraction efficiency of citrate containing buffer compositions of the present technology with silica-based and poly vinyl acetate (PVA)-based magnetic beads, assessed by PCR sensitivity and detection on real-time PCR platform (Savanna®, Quidel Corporation). Nucleotide extraction with citrate buffers of the present technology provides comparable extraction efficiency of SARS-CoV-2 with both PVA and silica-based magnetic beads, as assessed by PCR sensitivity and detection.

FIG. 11 illustrates Flu A nucleotide extraction efficiency of citrate containing buffer compositions of the present technology with silica-based and PVA-based magnetic beads, assessed by PCR sensitivity and detection on real-time PCR platform (Savanna®, Quidel Corporation). Nucleotide extraction with citrate buffers of the present technology provides comparable extraction efficiency of FluA with both PVA and silica-based magnetic beads, as assessed by PCR sensitivity and detection.

FIG. 12 illustrates Enterovirus nucleotide extraction efficiency of citrate containing buffer compositions of the present technology with silica-based and PVA-based magnetic beads, assessed by PCR sensitivity and detection on real-time PCR platform (Savanna®, Quidel Corporation). Nucleotide extraction with citrate buffers of the present technology provides better extraction efficiency of Enterovirus with silica-based magnetic beads compared to PVA-based magnetic beads, as assessed by PCR sensitivity and detection.

FIG. 13 illustrates Adenovirus nucleotide extraction efficiency of citrate containing buffer compositions of the present technology with silica-based and PVA-based magnetic beads, assessed by PCR sensitivity and detection on real-time PCR platform (Savanna®, Quidel Corporation). Nucleotide extraction with citrate buffers of the present technology provides comparable extraction efficiency of Adenovirus with both PVA and silica-based magnetic beads, as assessed by PCR sensitivity and detection.

FIG. 14 illustrates the extraction efficiency of citrate containing buffer compositions of the present technology for Fusobacterium necrophorum and Arcanobacterium haemolyticum.

FIG. 15 illustrates Streptococcus pyogenes (Strep A) detection sensitivity and specificity using citrate containing buffer compositions of the present technology.

FIG. 16 illustrates that comparable detection for Fusobacterium necrophorum is achieved using either a liquid specimen in ESwab™ transport media or a direct throat swab specimen using the citrate containing buffer compositions of the present technology.

In the figures, elements with the same or similar labels may refer to elements having the same or similar features, unless otherwise stated.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

In the field of nucleic acid detection, the ability extract and amplify target samples at an exponential rate using techniques, such as polymerase chain reaction (PCR) or an isothermal process such as helicase dependent amplification, has significantly increased the sensitivity of detection. However, requisite complicated and time-consuming steps involving multiple reagents and incubations, without contamination, has prevented the application of these techniques in compact, easy to use compositions, methods, assays, kits and instruments that produce accurate results in a limited amount of time.

### Citrate Lysis and Wash Buffers

The two-part buffer compositions, methods, assays, kits and kits of parts described herein, can be used for qualitative detection and/or differentiation of various and multiple analytes, such as target nucleotides which may be associated with, for example, a pathogen, microbe, bacteria, virus, fungus or other microorganism. For example, in some embodiments the buffer compositions described herein can be used to extract nucleotides from samples, such as virus containing samples, for downstream analyses by molecular biological techniques, including rapid multiplexed Real-Time PCR (RT-PCR) assays for the qualitative detection and differentiation of a nucleotide from a pathogen of interest.

The two-part (lysis and wash) buffer compositions of the present technology comprise novel formulations developed to effectively extract nucleic acids from various samples, such as the three significant virus types. For example, the buffer compositions of the present technology have utility in extracting nucleotides from viral types, including capsid viruses containing RNA (e.g., enterovirus, rhinovirus), enveloped viruses containing RNA (e.g., flu, SARS), and capsid viruses containing DNA (e.g., adenovirus).

The novel formulations of low pH lysis buffers and wash buffers, described herein, provide superb extraction efficiency and improved sensitivity of microbes, such as pathogenic viruses, including respiratory viruses (e.g., SARS, Flu A, Flu B, and RSV) when assessed by PCR. The novel lysis and wash buffer formulations described herein provide unexpectedly superior extraction efficiency in simplified/streamlined protocols without requiring hazardous reagents.

For example, the two-part buffer compositions describe herein provide superior extraction efficiency and detection sensitivity when compared to standard extraction buffer protocols as assessed by PCR based detection and sensitivity.

Additionally, the buffer compositions provided herein eliminate the need for costly and time-consuming sample extraction instruments. Standard nucleotide extraction techniques (e.g., NucliSENS® easyMAG®) require expensive and lengthy protocols to remove many inhibitory effects and extract nucleic acids. For example, extraction systems utilizing standard buffers and methods (e.g., NUCLISENS® EASYMAG®) require at least about 40 minutes while nucleotide extraction using the buffers and methods described herein can be completed in about 8 minutes, approximately 20% the time required by standard techniques. Standard buffer extraction protocols also require offline heat lysis extractions prior to PCR setup. Such time consuming and laborious steps are not needed in conjunctions with the extraction methods described herein utilizing the novel buffer compositions of the present technology.

Furthermore, the two-part lysis and wash buffer compositions of the present technology provide extraction of multiple nucleotide targets, such as multiple pathogenic respiratory virus nucleotides, from a single sample. Moreover, buffers, methods, assays and kits described herein provide extraction of such multiple nucleotide targets in a fast, sample-to-result workflow. For example, the buffer compositions described herein provide fast nucleic acid extraction methods, assays, systems and kits which allow completion of extraction protocols in less than about 25 minutes, such as about 24 minutes, about 23 minutes, about 22 minutes, about 21 minutes, about 20 minutes, or less. Accordingly, the buffers, methods, assays and systems described herein reduce hands-on time required by an end user performing the provided nucleotide extraction methods with the novel lysis and wash buffers. In some embodiments, end users simply add sample specimen into a cartridge comprising the buffers of the present technology, and the cartridge added into an instrument for automated extraction, washing, elution and amplification of nucleic acid, if present. Such cartridge and instrument platforms are well known and available commercially, exemplified by Savanna (Quidel Corporation), Prism (Applied Biosystems), Lightcycler (Roche), MX4000 (Strategene), SmartCycler (Cepheid), iCycler IQ (BioRad), etc.

Moreover, the buffer compositions described herein include non-hazardous ingredients that are amenable to convenient storage at standard temperatures. Accordingly, the buffer compositions provided here eliminate the need for hazard labels on and reduce shipping restrictions that are required in conjunction with production and distribution of standard extraction buffers. For example, buffers described herein may be stored at temperatures between about 2° C. to about 30° C. and in containers without hazard labels.

In some embodiments, the non-hazardous reagents that are present in the buffer compositions of the present technology comprise citrate and may be referred to as citrate lysis and/or wash buffers. Such buffers may include N-acetyl cysteine (NAC), polyoxyethylene acyl ether (Brij-58), sodium citrate, such as sodium citrate pH 2.5, aluminum potassium sulfate, potassium citrate and a polysorbate-type nonionic surfactant (Tween-20). For example, certain non-hazardous, low pH, citrate lysis buffers described herein comprise about 47.5 wt % N-acetyl cysteine (NAC), about 0.1 wt % polyoxyethylene acyl ether (Brij-58), about 1.5 wt % sodium citrate pH 2.5, and about 0.5 wt % aluminum potassium sulfate. Certain non-hazardous, low pH, citrate wash buffers described herein comprise about 0.5 wt % potassium citrate and about 0.01 wt % a polysorbate-type nonionic surfactant (Tween-20).

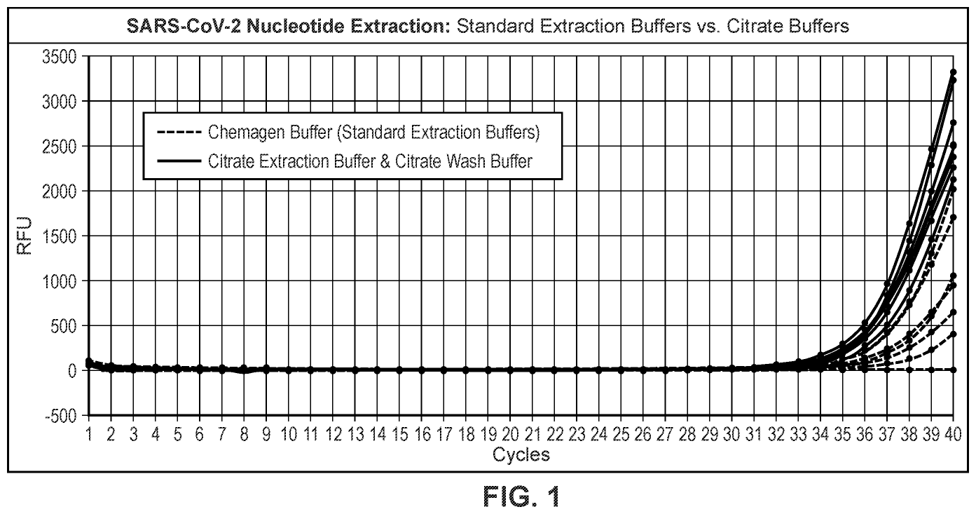

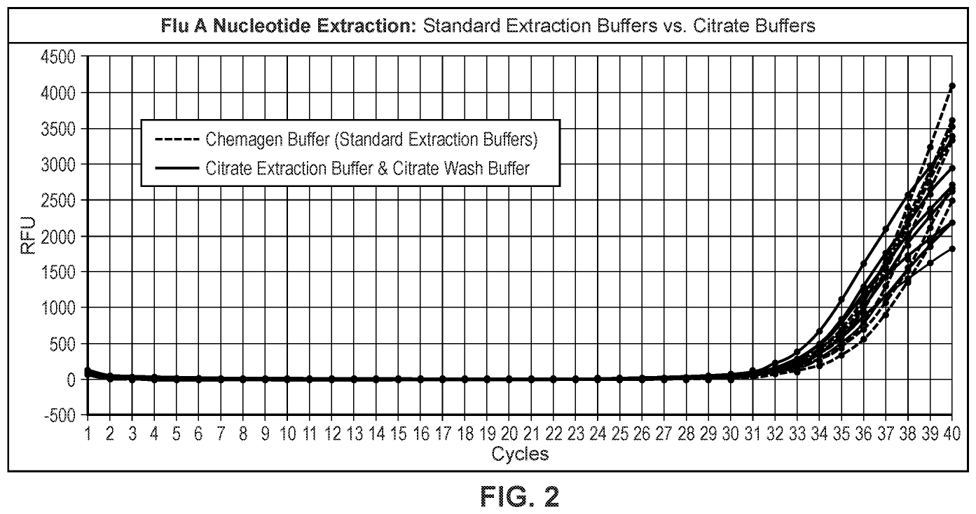

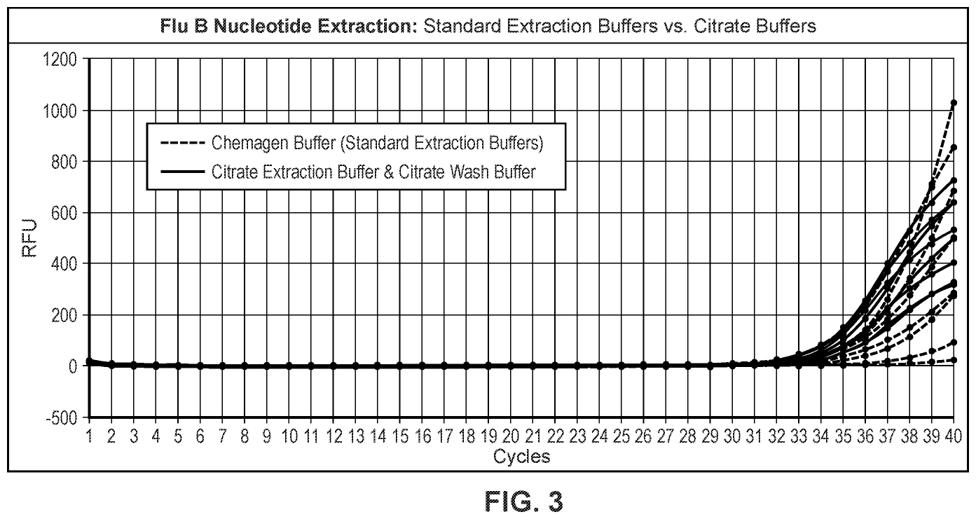

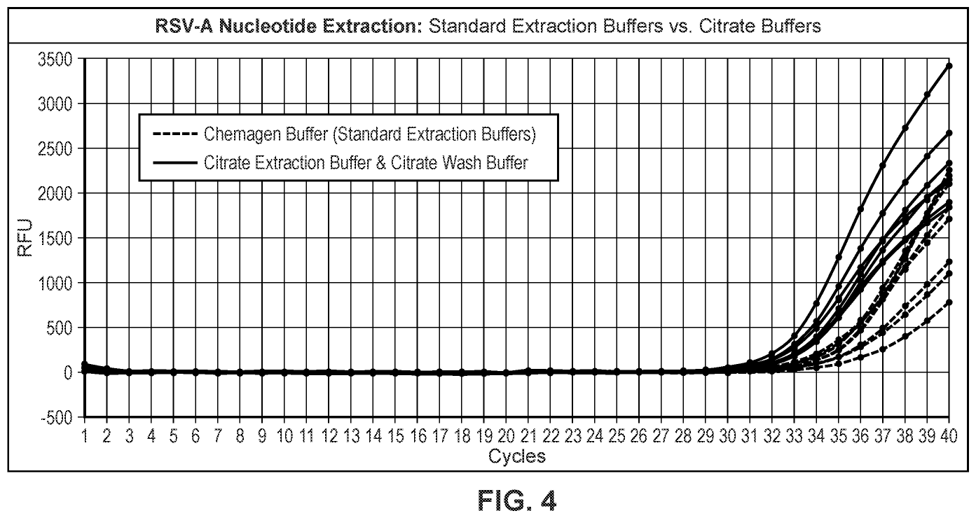

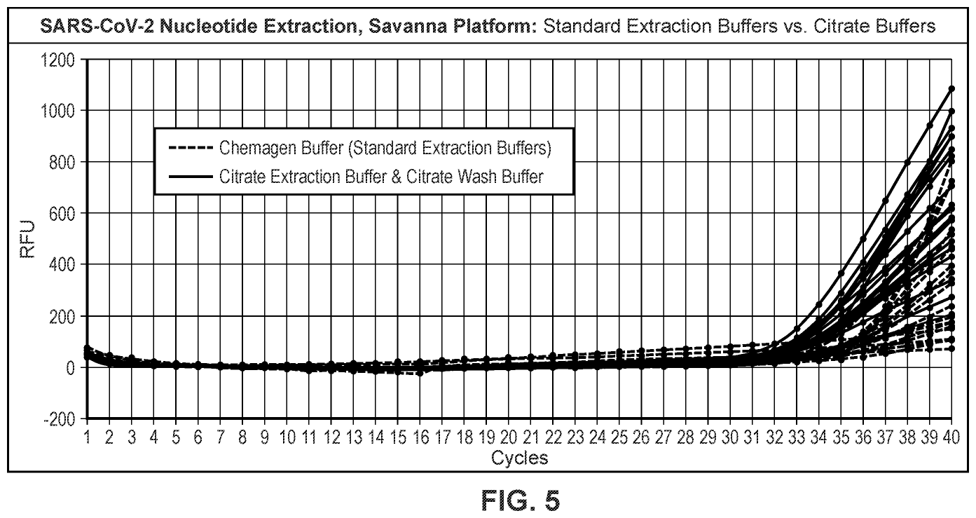

Unexpectedly, the citrate buffer compositions comprising such low pH, citrate lysis and wash buffers described herein achieve higher analytical sensitivities than standard extraction buffers as assessed by PCR detection sensitivity (FIGS. 1-5). Data demonstrating this improved sensitivity for various viral nucleotide extraction targets as shown in FIGS. 1-5 is summarized in Table 1.

TABLE 1
PCR Sensitivity of Current Citrate Buffers vs. Standard Buffers

| | | Analytical Sensitivity | | |
|---|---|---|---|---|
| | | Chemagen | Citrate | |
| | | Buffer | Lysis | |
| | | (Standard | Buffer & | |
| Sample | Viral | Extraction | Citrate | |
| ID | Strain | Buffers) | Wash Buffer | Improvement |
| SARS- | USA/WA1/ | 3.18E+01 | 9.56E+00 | Citrate buffers is 3 |
| CoV-2 | 2020 | | | times better |
| Flu | California/ | 4.17E+02 | 4.17E+01 | Citrate buffers is |
| A/H3N2 | 7/04 | | | 10 times better |
| Flu B | B/Panama/ | 1.27E+02 | 4.23E+01 | Citrate buffers is 3 |
| Yamagata | 45/90 | | | times better |
| RSV-A | 4/2015 | 1.39E+00 | 4.20E−01 | Citrate buffers is 3 |
| | Isolate 1 | | | times better |

The citrate buffer compositions comprising such low pH, citrate lysis and wash buffers described herein were also validated on clinical specimens using an automated cartridge and instrument platform (Savanna®, Quidel Corporation) and compared to standard, industry accepted protocols and buffers in order to establish and validate clinical performance of the buffers described herein. All clinical performance endpoints/acceptance criteria were met. This demonstrates the citrate buffers described herein provide fast, non-hazardous, and clinically reliable results when compared to standard buffers and previously described methods. The results from the clinical validation studies are summarized in Table 2.

TABLE 2
Clinical Validation of Current Low pH Citrate Buffers vs. Standard Buffers

| Virus | TP | FP | TN | FN | PPA | NPA |
|---|---|---|---|---|---|---|
| Influenza A | 5 | 0 | 219 | 0 | 100% (5/5) | 100% (219/219) |
| | | | | | 95% Cl = 56.6%-100% | 95% Cl = 98.3%-100% |
| Influenza B | 7 | 0 | 217 | 0 | 100% (7/7) | 100% (217/217) |
| | | | | | 95% Cl = 64.6%-100% | 95% Cl = 98.3%-100% |
| RSV | 22 | 0 | 202 | 0 | 100% (22/22) | 100% (202/202) |
| | | | | | 95% Cl = 85.1%-100% | 95% Cl = 98.1%-100% |
| SARS-CoV-2 | 143 | 0 | 80 | 1 | 100% (143/144) | 100% (80/80) |
| | | | | | 95% Cl = 96.2%-99.9% | 95% Cl = 95.4%-100% |

*total positive (TP); false positive (FP); total negative (TN); false negative (FN); positive percent agreement (PPA); negative percent agreement (NPA)

### Acetate, Glycine and PBS Plus 0.5M NaCl Lysis Buffers

In some embodiments, the non-hazardous reagents that are present in the buffer compositions of the present technology comprise acetate, glycine or PBS plus NaCl instead of a citrate component in the lysis buffer. Such buffers are referred to, in some embodiments, as low pH, acetate, glycine or PBS plus NaCl lysis buffers. In some embodiments these buffers comprise about 1.5 wt % acetate, about 1.5 wt % glycine or about 1.5 wt % PBS plus 0.5M NaCl, instead of about 1.5% sodium citrate. These buffers can be used in the same manner described above for citrate lysis buffers and can be used in conjunction with the wash buffers described herein. Such low pH acetate, glycine and/or PBS plus 0.5M NaCl lysis buffers may also include N-acetyl cysteine (NAC), polyoxyethylene acyl ether (Brij-58), aluminum potassium sulfate. For example, certain non-hazardous, low pH, lysis buffers described herein comprise about 47.5 wt % N-acetyl cysteine (NAC); about 0.1 wt % polyoxyethylene acyl ether (Brij-58); about 1.5 wt % sodium acetate pH 3.5; and about 0.5 wt % aluminum potassium sulfate. Other non-hazardous, low pH, lysis buffers described herein comprise about 47.5 wt % N-acetyl cysteine (NAC); about 0.1 wt % polyoxyethylene acyl ether (Brij-58); about 1.5 wt % glycine pH 3.2; and about 0.5 wt % aluminum potassium sulfate. Additional non-hazardous, low pH, lysis buffers described herein comprise about 47.5 wt % N-acetyl cysteine (NAC); about 0.1 wt % polyoxyethylene acyl ether (Brij-58); about 1.5 wt % PBS plus 0.5M NaCl pH 2.6; and about 0.5 wt % aluminum potassium sulfate.

In some embodiments, such lysis buffers can be used in conjunction with certain non-hazardous, low pH, citrate wash buffers described herein and, in some embodiments comprising about 0.5 wt % potassium citrate and about 0.01 wt % a polysorbate-type nonionic surfactant (Tween-20).

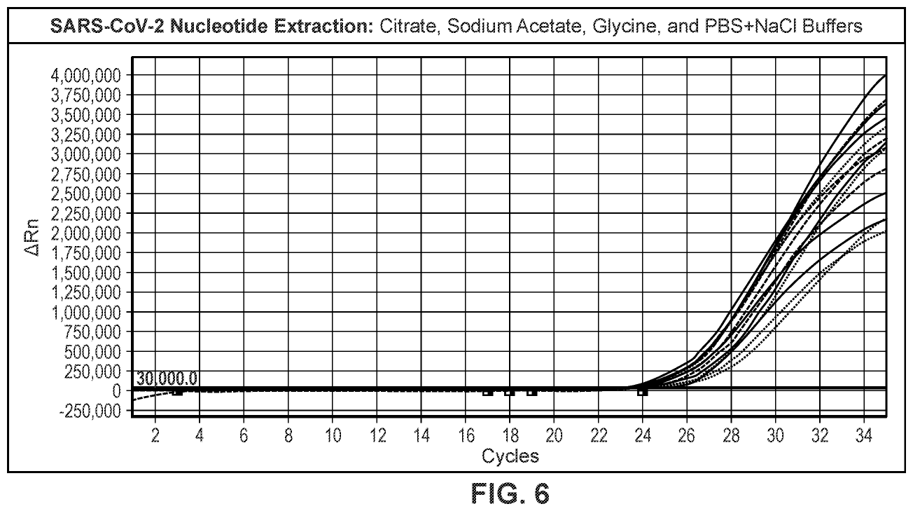

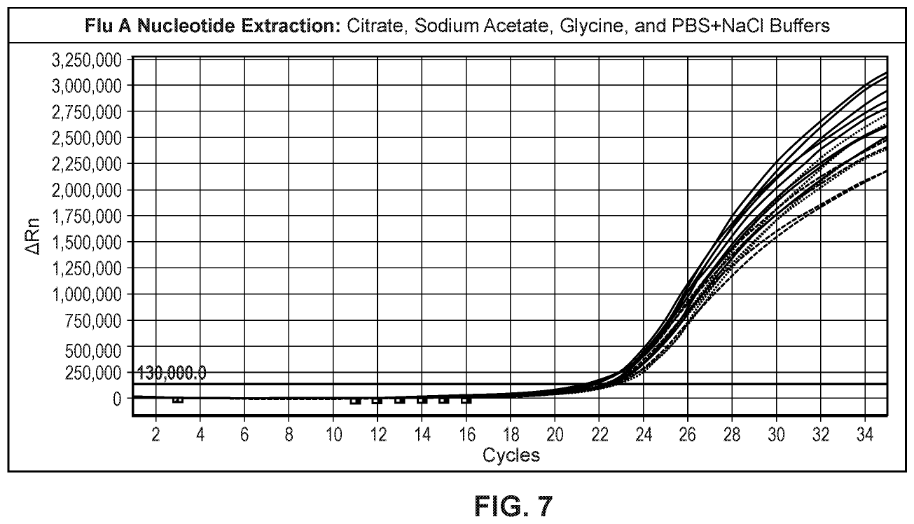

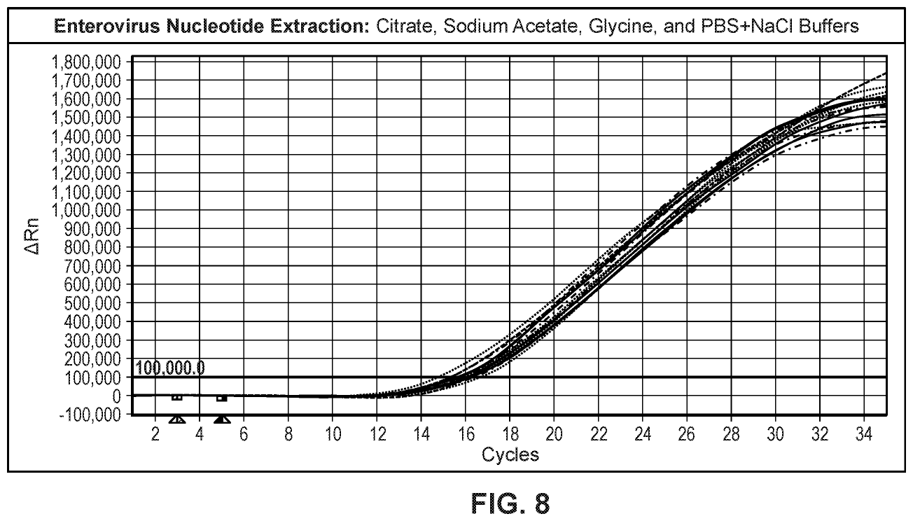

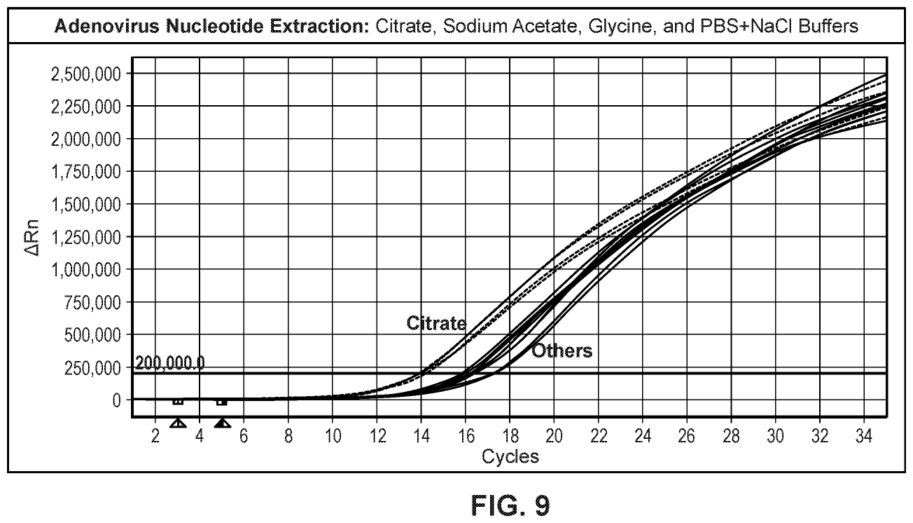

Acetate, glycine or PBS plus NaCl containing lysis buffers of the present technology may be used in the same fast, streamlined and non-hazardous nucleotide extraction methods and assays as described for citrate buffers where they provide similar performance and enhanced extraction efficiency when compared to standard buffers, as assessed by PCR based techniques for detection and sensitivity. The alternative low pH lysis buffers have comparable performance to the citrate buffers described herein, excluding Adenovirus which is 2.0 Cts faster with the citrate buffers compared to the other low pH lysis buffers. Accordingly, the acetate, glycine or PBS plus NaCl containing buffer compositions comprising described herein achieve similar analytical sensitivities as demonstrated for the citrate buffers provided herein, as assessed by PCR detection sensitivity (FIGS. 6-9). Ct (cycle threshold) data for various low pH, acetate, glycine or PBS plus NaCl lysis buffers shown in FIGS. 6-9 is presented in Table 3.

TABLE 3
Comparison of Citrate, Acetate, Glycine and PBS plus 0.5M NaCl Lysis Buffers

| | | | | Delta |
|---|---|---|---|---|
| Low pH Lysis | | AVG | STDEV | (Condition - |
| Buffer | Sample ID | Ct | Ct | Citrate) |
| Citrate | SARS at 9.60E+00 TCID50/mL | 33.2 | 0.6 | N/A |
| Sodium Acetate | SARS at 9.60E+00 TCID₅₀/mL | 32.8 | N/A | −0.3 |
| Glycine | SARS at 9.60E+00 TCID₅₀/mL | 33.6 | 0.5 | 0.4 |
| PBS + NaCl | SARS at 9.60E+00 TCID₅₀/mL | 33.7 | 1.1 | 0.5 |
| Citrate | Flu A at 1.40E+01 TCID50/mL | 32.4 | 0.2 | N/A |
| Sodium Acetate | Flu A at 1.40E+01 TCID₅₀/mL | 31.8 | 0.3 | −0.6 |
| Glycine | Flu A at 1.40E+01 TCID₅₀/mL | 32.5 | 0.3 | 0.1 |
| PBS + NaCl | Flu A at 1.40E+01 TCID₅₀/mL | 32.1 | 0.5 | −0.4 |
| Citrate | Enterovirus Type 71 at 3.80E+03 | 22.6 | 0.4 | N/A |
| | TCID₅₀/mL | | | |
| Sodium Acetate | Enterovirus Type 71 at 3.80E+03 | 23.0 | 0.4 | 0.4 |
| | TCID₅₀/mL | | | |
| Glycine | Enterovirus Type 71 at 3.80E+03 | 22.9 | 0.8 | 0.3 |
| | TCID₅₀/mL | | | |
| PBS + NaCl | Enterovirus Type 71 at 3.80E+03 | 23.2 | 0.9 | 0.5 |
| | TCID₅₀/mL | | | |
| Citrate | Adenovirus Type 11 at 1.95E+04 | 20.0 | 0.2 | N/A |
| | TCID₅₀/mL | | | |
| Sodium Acetate | Adenovirus Type 11 at 1.95E+04 | 22.5 | 0.1 | 2.4 |
| | TCID₅₀/mL | | | |
| Glycine | Adenovirus Type 11 at 1.95E+04 | 22.1 | 0.2 | 2.1 |
| | TCID₅₀/mL | | | |
| PBS + NaCl | Adenovirus Type 11 at 1.95E+04 | 22.8 | 0.6 | 2.8 |
| | TCID₅₀/mL | | | |

### Magnetic Beads

In some embodiments, the buffer compositions, methods, assays, kits and kits of parts described herein described herein can be used in conjunction with nanoparticles, such as magnetic nanoparticles and magnetic beads, for extraction of nucleotides from biological samples. For example, the low pH, non-hazardous buffers may be used in conjunction with magnetic beads for determining presence of certain nucleic acids, such as nucleic acids specific for certain microbes, in a sample of interest. The buffer compositions provided herein are compatible with various types of beads, including but not limited to silica and PVA-based magnetic beads.

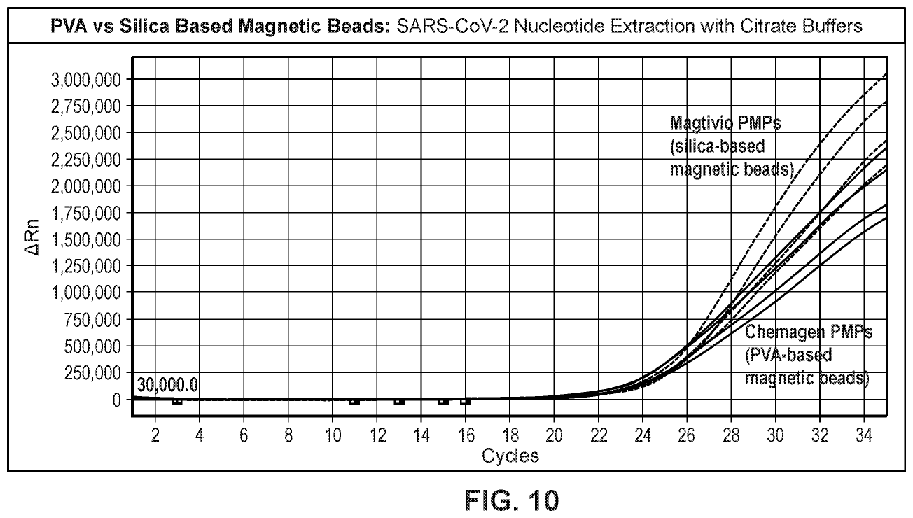

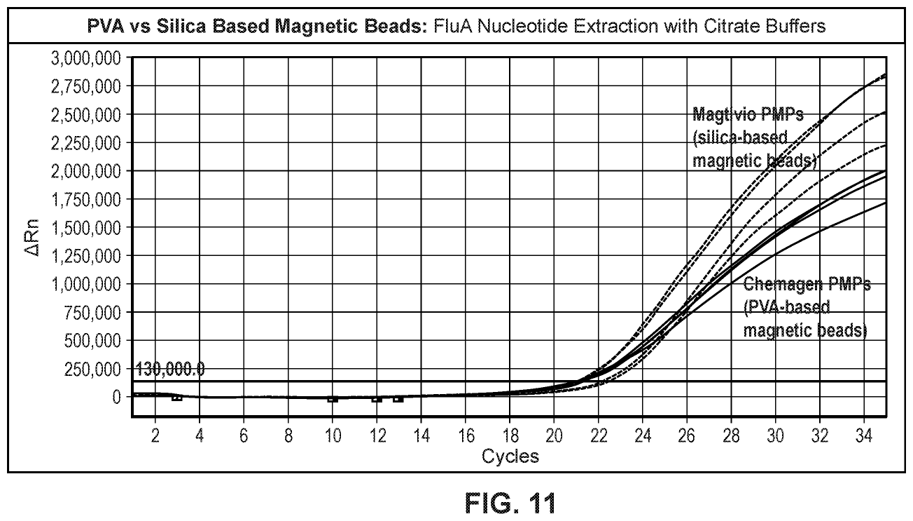

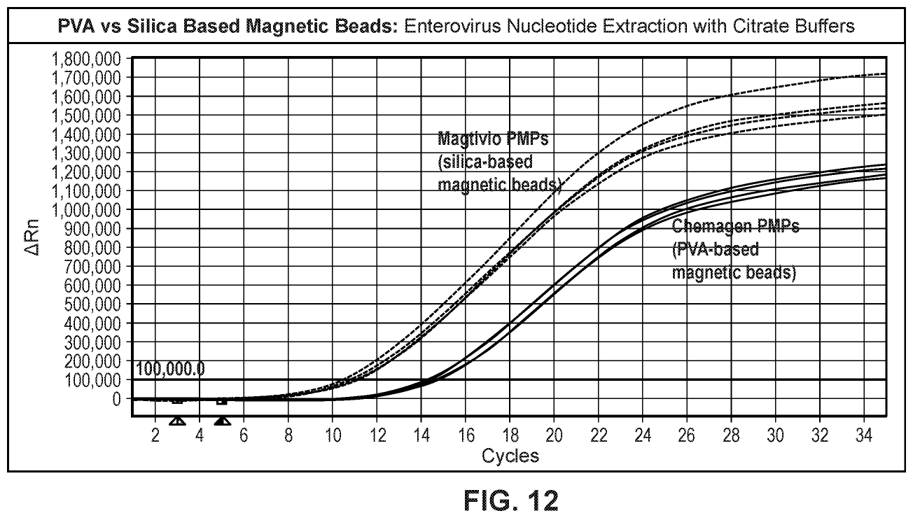

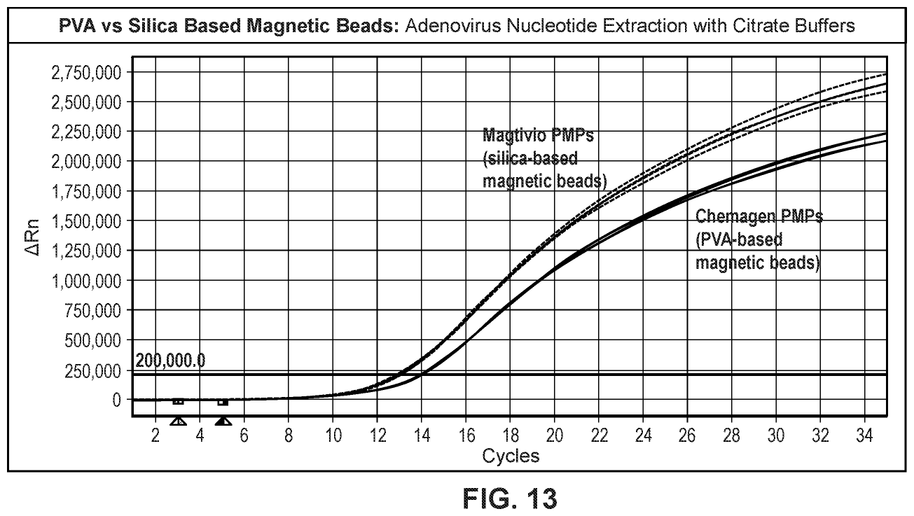

The low pH, non-hazardous buffers and methods described herein demonstrate efficient nucleotide extraction with both silica-based and PVA-based magnetic beads for extraction of target nucleotides which may be associated with, for example, a pathogen, microbe, bacteria, virus, fungus or other microorganism, such as viruses including but not limited to SARS-CoV-2, Flu A, enterovirus and adenovirus. Efficiency of the buffer compositions and methods described herein for extraction of various viral nucleotides with both silica and PVA-based magnetic beads is illustrated in FIGS. 10-13 and the underlying Ct (cycle threshold) data is presented in Table 4. The results show the buffer compositions, methods, assays, kits and kits of parts described herein are compatible with both silica-based and PVA-magnetic beads for efficient extraction of nucleotides from various sample types.

TABLE 4
Extraction from Silica and PVA Based Magnetic
Beads with Citrate Lysis Buffers

| | | | | Delta |
|---|---|---|---|---|
| Magnetic | | AVG | STDEV | (Condition - |
| Beads | Sample ID | Ct | Ct | CTRL) |
| PVA | SARS at 9.60E+00 | 32.3 | 0.7 | N/A |
| | TCID₅₀/mL | | | |
| Silica | SARS at 9.60E+00 | 33.2 | 1.7 | 0.9 |
| | TCID₅₀/mL | | | |
| PVA | SARS at 2.88E+01 | 29.8 | 1.1 | N/A |
| | TCID₅₀/mL | | | |
| Silica | SARS at 2.88E+01 | 30.9 | 0.8 | 1.1 |
| | TCID₅₀/mL | | | |
| PVA | Flu A at 1.40E+01 | 32.6 | 1.0 | N/A |
| | TCID₅₀/mL | | | |
| Silica | Flu A at 1.40E+01 | 33.0 | 1.1 | 0.4 |
| | TCID₅₀/mL | | | |
| PVA | Flu A at 4.20E+01 | 31.1 | 0.2 | N/A |
| | TCID₅₀/mL | | | |
| Silica | Flu A at 4.20E+01 | 31.6 | 0.8 | 0.6 |
| | TCID₅₀/mL | | | |
| PVA | Enterovirus Type 71 at | 24.6 | 0.2 | N/A |
| | 3.80E+03 TCID₅₀/mL | | | |
| Silica | Enterovirus Type 71 at | 20.9 | 0.3 | −3.7 |
| | 3.80E+03 TCID₅₀/mL | | | |
| PVA | Adenovirus Type 11 at | 23.9 | 0.0 | N/A |
| | 1.95E+04 TCID₅₀/mL | | | |
| Silica | Adenovirus Type 11 at | 22.9 | 0.1 | −1.0 |
| | 1.95E+04 TCID₅₀/mL | | | |

### Analytes

In some embodiments, the buffer compositions, methods, assays, kits and kits of parts described herein, can be used for qualitative detection and/or differentiation of various and multiple analytes, such as target nucleotides which may be associated with, for example, a pathogen, microbe, bacteria, virus, fungus or other microorganism. For example, in some embodiments the present technology is related to buffer compositions and methods for extraction of nucleotides that can be analyzed via downstream molecular biological techniques, including, but not limited to, Polymerase Chain Reaction (PCR) technologies. Such PCR based techniques that are compatible with nucleotides extracted with the buffers and methods describe herein include, rapid multiplexed Real-Time PCR (RT-PCR) assays for the qualitative detection and differentiation of nucleotides from pathogens of interest. In some embodiments, pathogens of interest may include microbes, bacteria, virus, fungus or other microorganisms and infectious agents. In embodiments, the pathogens of interest include viruses, such as, influenza A (Flu A), influenza B (Flu B), respiratory syncytial virus (RSV), SARS-CoV-2, adenoviruses and enteroviruses.

The buffer compositions, methods, assays, kits and kits of parts described herein, provide non-hazardous, fast and streamlined extraction of various nucleic acids of interest, such as DNA and/or RNA. In some embodiments, the nucleic acid of interest is a viral RNA extracted from nasal and nasopharyngeal swab in viral transport media. In some embodiments, analyzed specimens, including nasal and nasopharyngeal swabs, are from patients with signs and symptoms of respiratory viral infection.

The buffer compositions, methods, assays, kits and kits of parts described herein, also provide, in some embodiments, extraction of viral nucleic acids associated with viral diseases including, but are not limited to, Flu A, Flu B, RSV, SARS-CoV-2, adenoviruses and enteroviruses. The technology provided herein can provide extraction of nucleotides that can then be analyzed for information related to infection, such as viral infection, in humans in conjunction with clinical and epidemiological risk factors.

In some embodiments, the buffer compositions, methods, assays, kits and kits of parts described herein involves testing that is performed laboratory personnel, such as personnel in laboratories certified under the Clinical Laboratory Improvement Amendments of 1988 (CLIA), 42 U.S.C. § 263a, to perform moderate/high complexity tests. In other embodiments, the buffer compositions, methods, assays, kits and kits of parts described herein can be distributed and used in other settings, such as patient care settings outside of the clinical laboratory environment.

In some embodiments, the buffer compositions, methods, assays, kits and kits of parts described herein, provide extraction reagents for providing nucleic acids that can be analyzed for results indicating a positive or negative result for detection of a pathogen of interest, such as a bacteria, virus, fungus or other microbe or microorganism. In some embodiments, positive or negative results may be indicative of the presence of a viral infection, such the presence of Flu A, Flu B, RSV, SARS-CoV-2, enteroviruses and/or adenoviruses. In some embodiments, positive or negative results provided by the technology provided herein may be considered in coordination with clinical correlation of patient history and other diagnostic information that may be necessary to determine patient infection status. For example, a positive or negative result for one pathogen, such as a viral pathogen, does not rule out the possibility of additional infections, such as bacterial infections or co-infection with other viruses.

In some embodiments, the technology provided herein may provide extraction of nucleic acids from novel pathogens, such as, for example, a novel influenza virus. In such instances, specimens should be collected and handled according to proper safety, documentation and submission guidelines.

In some embodiments, the buffer compositions, methods, assays, kits and kits of parts described herein, provide extraction of nucleic acids that can be used for determination of the presence or absence of influenza viral nucleic acids in a sample, such as a nasal or nasopharyngeal swab from a patient suspected of pathogenic infection and/or disease. Influenza viruses are causative agents of highly contagious, acute, viral infections of the respiratory tract. Influenza viruses are immunologically diverse, single-stranded RNA viruses. There are three types of influenza viruses: A, B, and C. Type A viruses are the most prevalent and are associated with most serious epidemics. Type B viruses produce a disease that is generally milder than that caused by type A. Type C viruses have never been associated with a large epidemic of human disease. Both Type A and B viruses can circulate simultaneously, but usually one type is dominant during a given season. Every year in the United States, on average 5%-20% of the population contract influenza; more than 200,000 people are hospitalized from influenza complications; and, about 36,000 people die from influenza-related causes. Some people, such as adults 65 years of age and older, young children, and people with certain health conditions, are at high risk for serious influenza complications.

In some embodiments, the buffer compositions, methods, assays, kits and kits of parts described herein, provide extraction of nucleic acids that can be used for determination of the presence or absence of SARS-CoV-2 virus nucleic acids in a sample, such as a nasal or nasopharyngeal swab from a patient suspected of pathogenic infection and/or disease. SARS-CoV-2, also known as the COVID-19 virus, was first identified in Wuhan, Hubei Province, China December 2019. This virus, as with the novel coronavirus SARS-1 and MERS, is thought to have originated in bats, however the SARS-CoV-2 may have had an intermediary host such as pangolins, pigs or civets. The WHO declared that COVID-19 was a pandemic on Mar. 11, 2020, and human infection spread globally, with hundreds of thousands of confirmed infections and deaths. The median incubation time is estimated to be 5.1 days with symptoms expected to be present within 12 days of infection. The symptoms of COVID-19 are similar to other viral respiratory diseases and include fever, cough and shortness of breath.

In some embodiments, the buffer compositions, methods, assays, kits and kits of parts described herein, provide extraction of nucleic acids that can be used for determination of the presence or absence of Human respiratory syncytial virus (RSV) nucleic acids in a sample, such as a nasal or nasopharyngeal swab from a patient suspected of pathogenic infection and/or disease. RSV is a negative single-stranded RNA virus of the family Paramyxoviridae. RSV is the major cause of lower respiratory tract infection and hospital visits during infancy and childhood. In the United States, 60% of infants are infected during their first RSV season, and nearly all children will have been infected with the virus by 2-3 years of age. Of those infected with RSV, 2-3% will develop bronchiolitis, necessitating hospitalization. Natural infection with RSV induces protective immunity that wanes over time—possibly more so than other respiratory viral infections—and thus people can be infected multiple times. Sometimes an infant can become symptomatically infected more than once, even within a single RSV season. Severe RSV infections have increasingly been found among elderly patients.

In some embodiments, the buffer compositions, methods, assays, kits and kits of parts described herein, provide extraction of nucleic acids that can be used in conjunction with an approach for a single, disposable, self-contained assay cartridge with reagents for a nucleic acid extraction and amplification processes, such as real time PCR or other amplification technology, that is used in conjunction with an instrument to detect and differentiate target nucleotides in a sample that is inserted into the cartridge. In some embodiments, the target nucleotides that may be detected with the system, cartridge, and methods provided herein are nucleotides from pathogens and/or microbes, such as bacteria, virus or fungus. For example, in some embodiments, the extracted, analyzed, detected and/or differentiated nucleotides may comprise DNA and/or RNA, such as RNA from influenza A, influenza B, RSV, and/or SARS-CoV-2. Other exemplary target nucleotide analytes include those from Bordetella pertussis, Bordetella parapertussis, C. difficile, Group A β-hemolytic Streptococcus (Streptococcus pyogenes), pyogenic Group C/G (Streptococcus dysgalactiae), herpes simplex virus 1, herpes simplex virus 2, varicella-zoster virus, human metapneumovirus, trichomonas, human adenovirus, parainfluenza virus (PIV-1, PIV-2, and/or PIV-3), Fusobacterium necrophorum, and Arcanobacterium haemolyticum. The self-contained cartridge can comprise reagents for detection and/or differentiation of any one, two, three, four, five, six, seven, eight, nine, ten, eleven, twelve, or more target analytes.

In some embodiments, the buffer compositions, methods, assays, kits and kits of parts described herein, provide extraction of nucleic acids that can be used for determination of results indicating the presence of absence of multiple target nucleotides from analysis of a single patient sample, such as a nasal or nasopharyngeal swab. For example, in some embodiments, a single sample may be extracted and then analyzed in a single, disposable and self-contained cartridge for the presence or absence of multiple pathogenic targets in the single sample, such as multiple bacterial, viral, and/or fungal nucleotides, and/or mixtures and combinations of the same. In some embodiments, a single sample may be extracted in accordance with the buffers and methods described here, and then analyzed with a single assay or cartridge, for the presence of multiple viral nucleotides, wherein the viral targets comprise different viral RNA targets including, but not limited to, influenza A, influenza B, RSV, S ARS -CoV-2, adenovirus, and/or enterovirus.

In some embodiments, the buffer compositions, methods, assays, kits and kits of parts described herein, provide extraction of nucleic acids that can be used for determination of the presence or absence of RNA or DNA present from a sample, such as a nasal, nasopharyngeal, sputum or blood sample, obtained from a symptomatic patient. In some embodiments, the technology provided herein may perform the complete analysis including extraction, amplification and detection in less than less than about 1 hour, less than about 30 minutes, and/or about 20 minutes, including such as about 22 minutes.

In some embodiments, the buffer compositions, methods, assays, kits and kits of parts described herein, provide extraction of nucleic acids to produce a nucleic acid sample that can serve as the basis for further manipulation and analysis by other systems, assays, devices, methods and kits for molecular biological analysis. Such analysis, in some embodiments, is initiated by placing a patient sample, such as a sample collected on a swab placed in nasal passage or in the mouth or throat, in a transport media, such as a viral transport media, prior to extraction of nucleic acids with the extraction buffers (lysis and wash buffers) provided herein. In some embodiments, the transport media containing specimen sample extracted from the swab is transferred to a liquid sample addition port, or sample port, of a for extraction of nucleic acids with buffer compositions described herein to be carried out within the cartridge. In other embodiments, a sample may be present on a swab is directly inserted into a cartridge comprising extraction buffers (lysis and wash buffers) described herein for extraction and processing of the sample on the swab.

In some embodiments, a transport media is transferred to a sample port of a cartridge with a transfer pipette supplied as part of a kit with the cartridge comprising the extraction buffer compositions and two-part buffer compositions described herein. In some embodiments, a provided transfer pipette includes an overflow chamber and is configured to transfer and/or dispense a specific, fixed and known volume of a sample, such as a patient sample extracted from a nasal or nasopharyngeal swab specimen in a transport media. In some embodiments, a provided transfer pipette is configured to transfer and/or dispense a volume of sample of between about 50-2000 μL, 50-1000 μL, 100-500 μL, 150-400 μL, 175-350 μL, 200-300 μL, 225-275 μL or a sample, or about 150 μL, about 200 μL, about 250 μL, about 300 μL or about 350 μL of a sample.

In some embodiments, after a sample is introduced to the sample port of a cartridge, the port is closed and the cartridge is inserted into an instrument for initiation of sample processing, including nucleic acid extraction via methods described herein comprising the extraction buffers, and two-part buffer compositions, described herein. In some embodiments, as detailed herein, the sample is pushed out of the sample port by a lysis buffer, such as the lysis buffers described herein. In some embodiments, the lysis buffer also rehydrates a process control, such as a Escherichia virus MS2 (MS2) process control. In some embodiments, the sample and process control, together with particles or beads, such as magnetic particles, including silica and PVA-based magnetic beads, are moved into an extraction chamber of a cartridge as described herein. In some embodiments, the solution comprising the sample, optionally a process control, and a fluid (such as a lysis buffer or transport medium) is mixed in the extraction chamber, and cells or organisms in the sample are further lysed by the mixing. In an embodiment, the mixing is by sonication of the extraction chamber. In some embodiments, beads having sample DNA and/or RNA associated therewith are washed, for example, with the wash buffers described here, and the DNA and/or RNA is eluted from the beads. In some embodiments, a solution comprising the purified and/or isolated DNA and/or RNA is used to rehydrate a lyophilized master mix that comprises reagents for amplification of the DNA and/or RNA. In an embodiment, the solution with the isolated and/or purified DNA and/or RNA is moved from the extraction chamber into a plurality of reagent chambers, each reagent chamber in dedicated fluid communication with a detection chamber. Each reagent chamber comprises reagents for amplification and detection of a particular DNA or RNA target analyte. In this way, a cartridge with 2, 4, 6, 8, 10, 12 or any number of reagent chambers with dedicated detection chambers achieves multiplexing analysis of target nucleic acids from a single sample. In an embodiment, the cartridge comprises four reagent chambers each with a dedicated detection chamber, where each reagent chamber comprises reagents (e.g., primers, probes, enzymes, salts, sugars, etc.) for amplification and detection of a specific target analyte. In one embodiment, each reagent chamber comprises reagents (also referred to in the art as master mixes) for amplification and detection of one of a nucleic acid from a specific pathogen. In an embodiment, the pathogens are selected from influenza A, influenza B, RSV, SARS-CoV-2, adenoviruses and/or enteroviruses. In an embodiment, the pathogens are selected from influenza A, influenza B, RSV, SARS-CoV-2, Bordetella pertussis, Bordetella parapertussis, C. difficile, Group A β-hemolytic Streptococcus (Streptococcus pyogenes), pyogenic Group C/G (Streptococcus dysgalactiae), herpes simplex virus 1, herpes simplex virus 2, varicella-zoster virus, human metapneumovirus, trichomonas, human adenovirus, parainfluenza virus (PIV-1, PIV-2, and/or PIV-3), Fusobacterium necrophorum, and Arcanobacterium haemolyticum. In some embodiments, reagent in each reagent chamber is transported into a dedicated detection chamber, where amplification of target nucleic acid sequences can be performed. In some embodiments, amplification in the detection chamber may include Taq-man® multiplex real-time RT-PCR reactions that are carried out under optimized conditions generating amplicons for the targeted virus (if present) and the process control present in the sample. In an embodiment, each detection chamber is configured with an optical window for interrogation by an optics system in the instrument that receives the cartridge, for inspection of the amplicons in the detection chamber to determine presence or absence of particular labels, tags or detection reagents.

In some embodiments, each master mix contains primers and labeled probes, such as dual-labeled probes, unique for one, two, or more viral targets and/or a process control. In some embodiments, the probes are labeled, for example, with a fluorophore on one end and a quencher on the other end. In some embodiments, the master mix reagent comprises reagents for a reverse transcriptase step to produce cDNA of viral RNA, if present, and optimally an MS2 bacteriophage process control RNA. In some embodiments, a polymerase cleaves the probe bound to complementary DNA sequences during DNA amplification, separating the fluorophore from the quencher. In some embodiments, this cleavage generates an increase in fluorescent signal and if sufficient fluorescence is achieved, the sample is reported as positive for the detected target sequence. In some embodiments, the instrument that receives the cartridge also includes a user interface screen for controlling, monitoring and reading results, such as positive, negative, and/or invalid results for the presence of absence of the targeted nucleotide sequences.

### EXAMPLES

### Example 1

### Comparison of Low pH, Non-Hazardous, Two-Part Citrate Extraction Buffer to Standard Extraction Buffer

To determine efficiency of the low pH, non-hazardous extraction buffer compositions described herein, a buffer of the present technology was compared to a standard extraction buffer with respect to extraction efficiency as assessed by PCR detection and sensitivity. The two-part buffer composition of the present technology was utilized in accordance with the extraction protocols described herein and below, while the standard buffer was used in accordance with manufacturer's suggested protocol.

The buffer of the present technology that was utilized for this comparison is a non-hazardous, two-part, low pH, citrate buffer comprising a lysis buffer and a wash buffer. The lysis buffer included the components as shown below in Table 5.

TABLE 5
Non-hazardous, low pH, citrate, lysis buffer

| Reagent | wt % |
|---|---|
| N-Acetyl Cysteine (NAC) | 47.5% |
| polyoxyethylene acyl ether (Brij-58) | 0.1% |
| Sodium Citrate pH 2.5 | 1.5% |
| Aluminum Potassium Sulfate | 0.5% |

The wash buffer included the reagents as shown below in Table 6.

TABLE 6
Non-hazardous, low pH, citrate, wash buffer

| Reagent | wt % |
|---|---|
| Potassium Citrate pH 2.5 | 0.5% |
| a polysorbate-type nonionic surfactant | 0.01% |
| (Tween-20) | |

This two-part buffer composition of the current technology was compared to a standard extraction buffer system (Chemagen, Perkin Elmer®). The standard extraction buffer system includes hazardous materials such as a lysis buffer comprising 60-80 wt % guanidinium thiocyanate (acutely toxic); a binding buffer including 50-70 wt % ethanol (flammable), 30-50 wt % sodium perchlorate (acutely toxic), and 1-3 wt % acetic acid (skin corrosive) and two wash buffers including ethanol (30-50 wt % or 60-80 wt %) and sodium perchlorate (20-25 wt %). Details related to the hazardous, flammable, toxic and corrosive ingredients included in the standard extraction buffer system are available in material safety data sheets provided for the standard buffers by the manufacturer.

### Standard Buffer Protocol

The standard buffer was used in accordance with the manufacturer's suggested protocol. Briefly, the standard protocol included placing a nasal swab specimen in 3.0 mL of viral transport media. For example, viral transport media includes about 9.75 wt % Hank's Balanced Salt Solution, (HBSS), about 0.20 wt % gentamicin (50 mg/mL), about 0.20 wt % amphotericin B (250 μg/mL), about 2.00 wt % heat inactivated fetal bovine serum (FBS), and about 0.10 wt % phenol red solution (10 mg/mL). The swab was then vortexed in the viral transport media for 10 seconds. Next, 800 μL of the standard extraction buffer was produced by pre-mixing 200 μL standard lysis buffer with 600 standard binding buffer in a separate 1.5 mL tube. Then, 200 μL of vortexed liquid swab sample in viral transport media was added into the tube containing the 800 μL of the standard extraction buffer.

Magnetic beads were then diluted by adding 1.0 mL of stock magnetic beads +2.3 mL of MG Water, in accordance with manufacturer's instructions. 100 μL of diluted magnetic beads were then added to the sample/extraction buffer mixture and the sample was then incubated for 5 minutes at room temperature with vortexing every 30 seconds.

After the 5 minutes incubation, the tubes were centrifuged for 2 seconds, and the magnetic beads were collected on the side of the 1.5 mL tube with a magnetic rack. The supernatant was removed and discarded. 500 μL of wash buffer was added to the magnetic beads and the bead pellet was resuspended by pipetting (6 times). Magnetic beads were then again collected on the side of the tube using the magnetic rack and the supernatant was removed and discarded. 500 μL Wash buffer was added to the magnetic beads again and the bead pellet was resuspended by pipetting (6 times). Magnetic beads were again collected on the side of the tube using the magnetic rack and the supernatant was removed as completely as possible and discarded.

The magnetic bead pellet was then incubated at 56° C. for 1 minute and 180 μL elution buffer (1.0 wt % 1M Tris-HCl, pH8.0) was added to the beads, The beads were resuspended in elution buffer by pipetting (6-8 times). The resulting mixture was incubated at 56° C. for an additional 1 min after which the supernatant (eluted nucleic acid) was removed. Supernatant eluates were collected in 1.5 mL tubes, then analyzed by PCR.

### Citrate Buffer Protocol

The two-part citrate buffer composition of the present technology was used in accordance with extraction protocols and methods of the present technology. Briefly, a nasal swab specimen was placed in 3.0 mL of viral transport media. The swab was vortexed in transport media for 10 seconds. 800 μL of the non-hazardous, low pH, citrate, lysis buffer from Table 5 was added into a 1.5 mL tube. 200 μL of the vortexed, liquid swab sample was added into the tube containing the citrate extraction buffer. 100 μL of diluted magnetic beads was added to each sample and the mixture was incubated for 5 minutes at 56° C. and vortexed every minute. After the 5-minute incubation, the tubes were centrifuged for 2 seconds, and the magnetic beads were collected on the side of a 1.5 mL tube using a magnetic rack. The supernatant was removed and discarded.

500 μL of the non-hazardous, low pH, citrate, wash buffer of Table 6 was added to the magnetic beads and the bead pellet was resuspended in the citrate wash buffer by vortexing for 5-10 seconds. The tubes were centrifuged for 2 seconds, magnetic beads were collected on the side of the tube using a magnetic rack and the supernatant (citrate wash buffer) was removed and discarded.

180 μL elution buffer was added to the beads and the beads were resuspended in elution buffer by pipetting (6-8 times). The elution buffer mixture was then incubated at 56° C. for 1 min after which the supernatant (eluted nucleic acid) was removed. Supernatant eluates were collected in 1.5 mL tubes, then analyzed by PCR.

Conclusions: Comparison of the PCR results for the two-part citrate buffer compositions described herein with the results achieved with the standard buffers demonstrate that the compositions and methods described herein achieve higher analytical sensitivities than standard extraction buffers as illustrated in FIGS. 1-5.

### Example 2

### Clinical Validation of Non-Hazardous, Low pH Citrate Buffers

To establish clinical performance of the two-part buffer compositions provided herein, the buffers were used in an assay for molecular detection and differentiation of four respiratory viruses, influenza A, influenza B, respiratory syncytial virus, and SARS-CoV-2, on a real-time PCR instrument (Savanna® Quidel Corporation). For comparison, also tested were the BioFire® Respiratory Panel 2.1 (RP2.1) (FDA cleared and CE marked), Lyra RSV+hMPV Assay (FDA cleared and CE marked) with 1) left-over archived respiratory specimens in transport medium and 2) left-over prospectively provided specimens in transport medium. The buffers used in this study were the non-hazardous, low pH, citrate, lysis and wash buffers of Tables 5 and 6.

The assay run on the Savanna instrument is real-time RT-PCR assay intended for the in vitro qualitative detection of influenza A, influenza B, RSV, and human coronavirus SARS-CoV-2 from viral RNA (referred to herein as RVP4 Assay) extracted from nasal, nasopharyngeal swab specimens in transport medium from patients with signs and symptoms of virus respiratory disease. All testing in this study was conducted with the Savanna Instrument.

The study was conducted in the United States and followed all laws and regulations for conducting clinical studies. Institutional Review Board (IRB) approval was obtained prior to commencement of the prospective field arm of the protocol. Informed consent was not required from patients prior to use of their specimens in the study or any study-specific procedures being done, as the U.S. FDA currently does not enforce informed consent for leftover, deidentified specimens. The study was conducted in a manner consistent with patient confidentiality as set forth in the Health Insurance Portability and Accountability Act (HIPAA) Authorization form.

Two hundred twenty-four (224) anterior nares or nasopharyngeal swab specimens in transport medium were obtained from fourteen (14) locations. Two hundred four (204) were archived frozen specimens. Twenty (20) were prospectively collected fresh specimens tested within 1-8 days post-collection (study on-going). Transport media that was used included universal transport medium (Copan UTM/VTM) and transport medium from(Quidel Corporation (Quidel Transport Media, QTM). Prospective specimens were transported to the testing site overnight on either dry ice or cold packs. The specimens were brought to room temperature and tested concurrently. The two-sided 95% confidence interval (95% CI) of the percent agreements (PPA/NPA) were determined using the Wilson-Score method.

The RVP4 assay comprising the two-part, non-hazardous, low pH citrate lysis and wash buffers of the present technology (Tables 5 and 6) demonstrated excellent total positive and negative percent agreement (100.0%) for the detection of influenza A, influenza B and RSV when compared to the BioFire® Respiratory Panel 2.1 Assay. The RVP4 Assay demonstrated excellent total positive percent agreement (100.0%) for the detection of RSV when compared to the BioFire® Respiratory Panel 2.1 Assay.

The RVP4 Assay comprising the two-part, non-hazardous, low pH citrate lysis and wash buffers of the present technology (Tables 5 and 6) demonstrated excellent total positive and negative percent agreement (99.3%) for the detection of SARS-CoV-2 when compared to the BioFire® Respiratory Panel 2.1 Assay. The RVP4 Assay comprising the two-part, non-hazardous, low pH citrate lysis and wash buffers of the present technology (Tables 5 and 6) demonstrated excellent total negative percent agreement (100.0%) for the detection of SARS-CoV-2 when compared to the BioFire® Respiratory Panel 2.1 Assay. All clinical performance endpoints/acceptance criteria were met as summarized in Table 2.

### Example 3

### Comparison of Various Non-Hazardous, Low pH Buffers

To determine efficiency of other low pH, non-hazardous extraction buffer compositions described herein, buffer of the present technology were compared to the non-hazardous, low pH, citrate lysis buffer described above in Example 1 (Table 5) and used in the clinical validation of Example 2. The other extraction buffers were compared to the citrate buffer with respect to extraction efficiency as assessed by PCR detection and sensitivity. The two-part buffer compositions of the present technology were utilized in accordance with the extraction protocols described in Example 1. The additional lysis buffers were tested in a two-part buffer compositions with the citrate wash buffer of Example 1 (Table 6)

The lysis buffers of the present technology that were analyzed in this comparison are non-hazardous, low pH, acetate, glycine or PBS+NaCl buffers, used with the wash buffer of Table 6 and compared to the lysis buffer of Table 5. The lysis buffers included this comparison included the components as shown below in Tables 7-9.

TABLE 7
Non-hazardous, low pH, acetate, lysis buffer

| Reagent | wt % |
|---|---|
| N-Acetyl Cysteine (NAC) | 47.5% |
| polyoxyethylene acyl ether (Brij-58) | 0.1% |
| Sodium Acetate pH 3.5 | 1.5% |
| Aluminum Potassium Sulfate | 0.5% |

TABLE 8
Non-hazardous, low pH, glycine, lysis buffer

| Reagent | wt % |
|---|---|
| N-Acetyl Cysteine (NAC) | 47.5% |
| polyoxyethylene acyl ether (Brij-58) | 0.1% |
| Glycine pH 3.2 | 1.5% |
| Aluminum Potassium Sulfate | 0.5% |

TABLE 9
Non-hazardous, low pH, PBS plus 0.5M NaCl, lysis buffer

| Reagent | wt % |
|---|---|
| N-Acetyl Cysteine (NAC) | 47.5% |
| polyoxyethylene acyl ether (Brij-58) | 0.1% |
| PBS plus 0.5M NaCl pH 2.6 | 1.5% |
| Aluminum Potassium Sulfate | 0.5% |

Conclusions: Comparison of the PCR results for the acetate, glycine and PBS plus NaCl lysis buffer compositions with the results achieved with the citrate lysis buffers demonstrate that these additional non-hazardous, low pH lysis buffers achieve comparable analytical sensitivities as the citrate lysis buffer as illustrated in FIGS. 6-9 and Table 3, with the caveat that adenovirus appears to be preferentially extracted with the citrate lysis buffer. Specifically, the observed PCR results are 2.0 cycle thresholds (Cts) faster for adenovirus detection with citrate lysis buffer, indicating adenovirus detection is 6.7 times more sensitive with citrate lysis buffer compared to the other tested buffers. Accordingly, the acetate, glycine and PBS plus NaCl lysis buffer compositions and methods utilizing the same provide improved extraction efficiency when compared to standard extraction buffers and methods.

### Example 4

### Non-Hazardous, Low pH Buffers Extraction Efficiency with Silica-Based and PVA-Based Magnetic Beads

To investigate the ability of the buffer compositions described herein to extract nucleic acid in conjunction with methods employing multiple types of magnetic beads, a buffer of the present technology was used for nucleic acid extraction with both silica-based (e.g., Magtivio silica-based paramagnetic particles) and PVA-based (e.g., Chemagen PVA-based paramagnetic particles) magnetic beads. Extraction efficiency was assessed by PCR detection and sensitivity. The two-part buffer composition of the present technology was utilized in accordance with extraction protocols of the present technology as described in Example 1, and performed side by side with the two different types of tested magnetic beads (silica and PVA).

The buffer of the present technology that was utilized for this comparison is the non-hazardous, two-part, low pH, citrate buffer composition comprising the lysis buffer of Table 5 and the wash buffer of Table 6.

Conclusions: Comparison of the PCR results for silica and PVA-based magnetic beads extracted with the citrate buffer composition, demonstrates that both types of magnetic beads are compatible with buffers of the present technology as illustrated in FIGS. 10-13 and Table 4. Comparable performance was observed for SARS-CoV-2, Flu A, and Adenovirus. Enterovirus is 3.0 cycle thresholds (Cts) faster with the silica-based magnetic beads indicating enterovirus detections is 10 times more sensitive with the silica-based magnetic beads compared to the PVA-based magnetic beads.

### Example 5

### Clinical Detection of Fusobacterium Necrophorum

Currently, there are no molecular assays on the market that detects Fusobacterium necrophorum and Arcanobacterium haemolyticum.

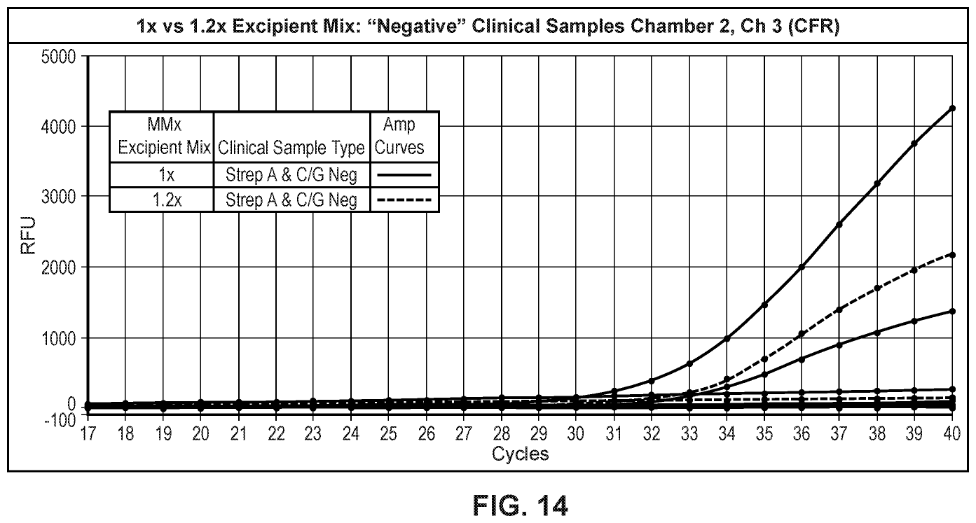

To detect the presence of Fusobacterium necrophorum and Arcanobacterium haemolyticum in a sample, a qualitative nucleic acid multiplex in vitro diagnostic test described herein was used to assess throat swab specimens obtained from patients exhibiting signs and symptoms of pharyngitis that tested negative for Strep A and Strep C/G. The test was also conducted to establish preliminary clinical performance using the citrate buffers described herein and a time-to-result (TTR) of 17 minutes. The results are described in FIG. 14.

Conclusions: The results demonstrate that the citrate buffers described herein, in a workflow of 17 minutes, could effectively extract and positively identified two clinical specimens for Fusobacterium necrophorum.

### Example 6

### Clinical Detection of Streptococcus Pyogenes (Strep A)

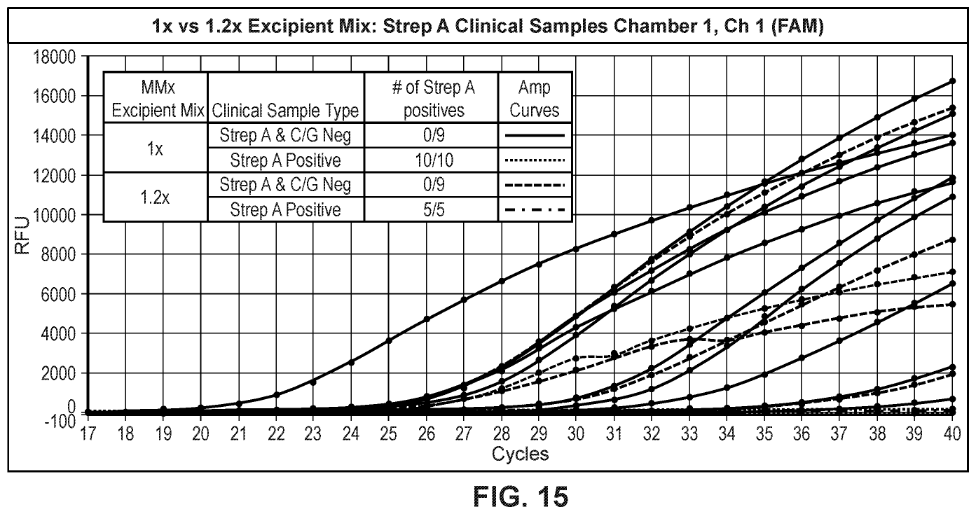

To detect the presence of Strep A in a sample, the qualitative nucleic acid multiplex in vitro diagnostic test described herein was used to establish preliminary clinical performance using the citrate buffers described herein and a time to result of 17 minutes. The results are described in FIG. 15.

Conclusions: The results demonstrate the citrate buffers described herein, in a workflow of 17 minutes time to result (TTR) has superb preliminary Strep A clinical performance. Using the citrate buffers of the present invention, 100% sensitivity and 100% specificity were achieved for a total of 19 samples. The results are described in FIG. 15 and in Table 10 below (where “Total” is the total number of samples tested; TP is total positive samples; FP is number of false positives; TN is total number of negative samples; and FN is number of false negatives).

TABLE 10
Preliminary Clinical Performance for Strep A detection

| | | | | | | | Sensitivity/ | Specificity/ |
|---|---|---|---|---|---|---|---|---|
| Organism | Type | Total | TP | FP | TN | FN | (PPA %) | (NPA %) |
| Strep A | Total | 19 | 10 | 0 | 9 | 0 | 100% (10/10) | 100% (9/9) |

### Example 7

### Evaluation of Liquid and Direct Swab Specimens

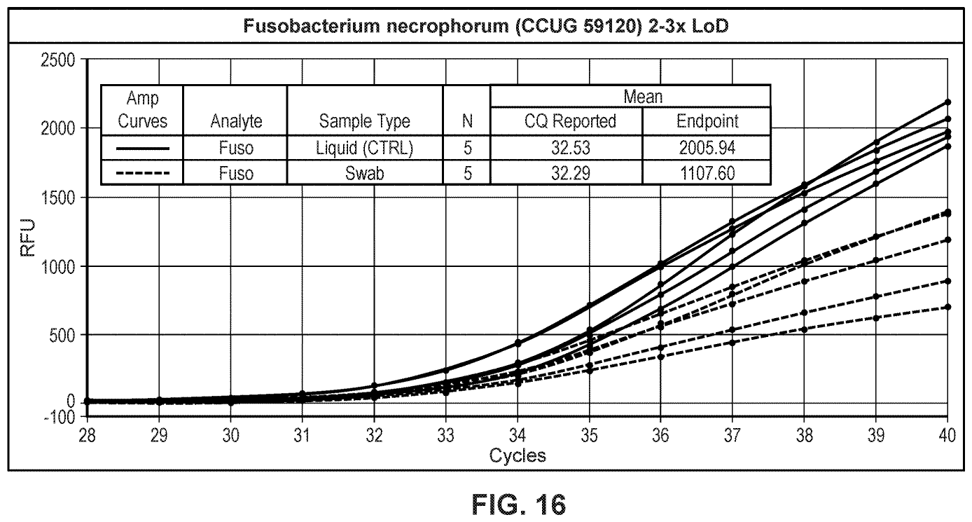

A further assay was conducted to determine whether comparable detection of Fusobacterium necrophorurn is achieved using either (i) a liquid specimen in ESwab™ transport media or (ii) a direct swab specimen in the citrate buffer described herein. The results are described in FIG. 16.

Conclusions: Comparable Fusobacterium necrophorum detection was achieved using ESwab™ transport media and a direct swab specimen using the citrate buffer described herein in the workflow of 17 minutes time to result.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

1. A two-part buffer composition, comprising:
(i) a first container comprising a lysis buffer comprising a lysis reagent, an antioxidant, and a detergent, the lysis buffer having a pH of about 1.8 to about 3.0; and
(ii) a second container comprising a wash buffer comprising a wash reagent and a detergent, the wash buffer having a pH of about 6.8 to about 7.6;
wherein the two part buffer composition comprises a nucleic acid extraction buffer system.

2. The two-part buffer composition of claim 1, wherein the lysis reagent is a citrate compound, an acetate compound, or a glycine compound.

3. The two-part buffer composition of claim 1, wherein the lysis reagent is sodium citrate, sodium acetate, or glycine.

4. The two-part buffer composition of claim 1, wherein the lysis reagent is sodium citrate, sodium acetate, or glycine, present in the lysis buffer in a range of between about 0.5-2.5 wt %.

5. The two-part buffer composition of claim 1, wherein the lysis reagent comprises phosphate buffered saline (PBS) S) and sodium chloride (NaCl).

6. The two-part buffer composition of claim 1, wherein the lysis reagent comprises PBS and 0.5M NaCl at a pH of between about 2.4-2.8.

7. The two-part buffer composition of claim 1, wherein the antioxidant is n-acetyl cysteine (NAC).

8. The two-part buffer composition of claim 1, wherein the antioxidant is NAC present in the lysis buffer in a range of between about 40-55 wt %.

9. The two-part buffer composition of claim 1, wherein the detergent in the lysis buffer is a non-ionic surfactant.

10. The two-part buffer composition of claim 1, wherein the detergent in the lysis buffer is a non-ionic polyoxyethylene surfactant, present in the lysis buffer in a range of between about 0.025-0.2 wt %.

11. The two-part buffer composition of claim 1, wherein the lysis buffer further comprises aluminum.

12. The two-part buffer composition of claim 1, wherein the lysis buffer further comprises aluminum potassium sulfate.

13. The two-part buffer composition of claim 1, wherein the wash reagent is a citrate compound.

14. The two-part buffer composition of claim 1, wherein the wash reagent is potassium citrate.

15. The two-part buffer composition of claim 1, wherein the wash reagent is potassium citrate present in the wash buffer in a range of between about 0.1-1.5 wt % or between about 0.25-1.0 wt %.

16. The two-part buffer composition of claim 1, wherein the wash detergent comprises a polysorbate surfactant (a Tween®).

17. The two-part buffer composition of claim 1,
wherein the lysis reagent comprises sodium citrate, sodium acetate, glycine, or PBS plus NaCl;
wherein the antioxidant comprises NAC;
wherein the lysis detergent comprises polyoxyethylene glycol hexadecyl ether (Brij®-58);
wherein the lysis buffer further comprises aluminum potassium sulfate;
wherein the wash reagent comprises a citrate reagent; and
wherein the wash detergent comprises a Tween®.

18. The two-part buffer composition of claim 1,
wherein the lysis reagent comprises about 1.5 wt % sodium citrate, about 1.5 wt % sodium acetate, about 1.5 wt % glycine pH 3.2, or about 1.5 wt % PBS plus NaCl pH 2.6;
wherein the antioxidant comprises about 47.5 wt % NAC;
wherein the lysis detergent comprises about 0.1 wt % polyoxyethylene glycol hexadecyl ether;
wherein the lysis buffer further comprises about 0.5 wt % aluminum potassium sulfate;
wherein the wash reagent comprises about 0.5 wt % potassium citrate; and
wherein the wash detergent comprises about 0.01 wt % Tween-20.

19. The two-part buffer composition of claim 1, wherein the nucleic acid extraction buffer system comprises magnetic particles.

20. A method for extracting nucleic acid from a sample, comprising:
i. providing a sample suspected of comprising nucleic acid;
ii. contacting the sample with a lysis buffer having a pH of about 1.8 to about 3.0, the lysis buffer comprising a lysis reagent, an antioxidant, and a lysis detergent, to produce a lysed sample comprising extracted nucleic acid, if present;
iii. contacting the lysed sample with a plurality of magnetic particles to produce magnetic particles complexed with the extracted nucleic acid, if present;
iv. exposing the magnetic particles complexed with the extracted nucleic acid, if present, with a magnetic field and removing the lysis buffer;
v. contacting the magnetic particles complexed with the extracted nucleic acid, if present, with a wash buffer having a pH of about 6.8 to about 7.6, the wash buffer comprising a wash reagent and a wash detergent, to produce a washed sample;
vi. exposing the washed sample comprising magnetic particles complexed with the nucleic acid, if present, with a magnetic field and removing the wash buffer to produce magnetic particles complexed with washed nucleic acid, if present;
vii. contacting the magnetic particles complexed with washed nucleic acid, if present, with an elution buffer to produce an elution sample comprising washed, extracted nucleic acid, if present;
viii. exposing the elution sample comprising washed, extracted nucleic acid, if present, to a magnetic field to remove the magnetic particles;
ix. contacting the eluted, washed, extracted nucleic acid, if present, with reagents for amplification of the nucleic acid; and
x. amplifying the nucleic acid, if present, and detecting its amplification products.

21. The method of claim 20, wherein the lysis reagent comprises a citrate reagent, an acetate reagent, or a glycine reagent.

22. The method of claim 20, wherein the lysis reagent comprises sodium citrate, sodium acetate, or glycine.

23. The method of claim 20, wherein the lysis reagent comprises about 1.5 wt % sodium citrate, sodium acetate, or glycine.

24. The method of claim 20, wherein the lysis reagent comprises phosphate buffered saline (PBS) and sodium chloride (NaCl).

25. The method of claim 20, wherein the lysis reagent comprises PBS and 0.5M NaCl at pH 2.6.

26. The method of claim 20, wherein the antioxidant comprises n-acetyl cysteine (NAC).

27. The method of claim 20, wherein the lysis detergent comprises polyoxyethylene glycol hexadecyl ether (Brij-58).

28. The method of claim 20, wherein the lysis buffer further comprises aluminum.

29. The method of claim 20, wherein the lysis buffer further comprises aluminum potassium sulfate.

30. The method of claim 20, wherein the wash reagent comprises a citrate reagent.

31. The method of claim 20, wherein the wash reagent comprises potassium citrate.

32. The method of claim 20, wherein the wash detergent comprises a polysorbate surfactant.

33. The method of claim 20,
wherein the lysis reagent comprises sodium citrate, sodium acetate, glycine, or PBS plus NaCl;
wherein the antioxidant comprises NAC;
wherein the lysis detergent comprises polyoxyethylene glycol hexadecyl ether (Brij®-58);
wherein the lysis buffer further comprises aluminum potassium sulfate;
wherein the wash reagent comprises a citrate reagent; and
wherein the wash detergent comprises a polysorbate-type nonionic surfactant.

34. The method of claim 20,
wherein the lysis reagent comprises about 1.5 wt % sodium citrate, about 1.5 wt % sodium acetate, about 1.5 wt % glycine pH 3.2, or about 1.5 wt % PBS plus NaCl pH 2.6;
wherein the antioxidant comprises about 47.5 wt % NAC;
wherein the lysis detergent comprises about 0.1 wt % polyoxyethylene glycol hexadecyl ether;
wherein the lysis buffer further comprises about 0.5 wt % aluminum potassium sulfate;
wherein the was reagent comprises about 0.5 wt % potassium citrate; and
wherein the wash detergent comprises about 0.01 wt % polysorbate-type nonionic surfactant.

35. The method of claim 20, wherein the method comprises a cartridge and an instrument for automated extraction, washing, elution and amplification of nucleic acid, if present.

36. A kit comprising the two-part buffer composition of claim 1 and user instructions for extracting, washing and eluting nucleic acid from a sample with said two-part buffer composition.

37. The kit of claim 36, further comprising a container and a pipette.
